(19) United States
(12) Patent Application Publication
(10) Pub. No.: US 2022/0354619 A1
(43) Pub. Date: Nov. 10, 2022

(54) METHODS OF INITIATING ALIGNER-BASED ORTHODONTIC TREATMENT OF A PATIENT

(71) Applicant: Christopher C. Cosse, Shreveport, LA (US)

(72) Inventor: Christopher C. Cosse, Shreveport, LA (US)

(21) Appl. No.: 17/736,321

(22) Filed: May 4, 2022

Related U.S. Application Data
(60) Provisional application No. 63/185,806, filed on May 7, 2021.
(60) Provisional application No. 63/184,374, filed on May 5, 2021.

(51) Int. Cl. A61C 7/00; A61C 7/08; A61C 9/00

(52) U.S. Cl. CPC A61C 7/002; A61C 7/08; A61C 9/0053

(57) ABSTRACT

Methods of initiating aligner-based orthodontic treatment of a patient. A method of initiating aligner-based orthodontic treatment of a patient includes recording a dental condition data set, producing one or more dental aligners, and providing the one or more dental aligners to the patient. At least the producing the one or more dental aligners and the providing the one or more dental aligners to the patient are performed at a service provider office while the patient remains at the service provider office.

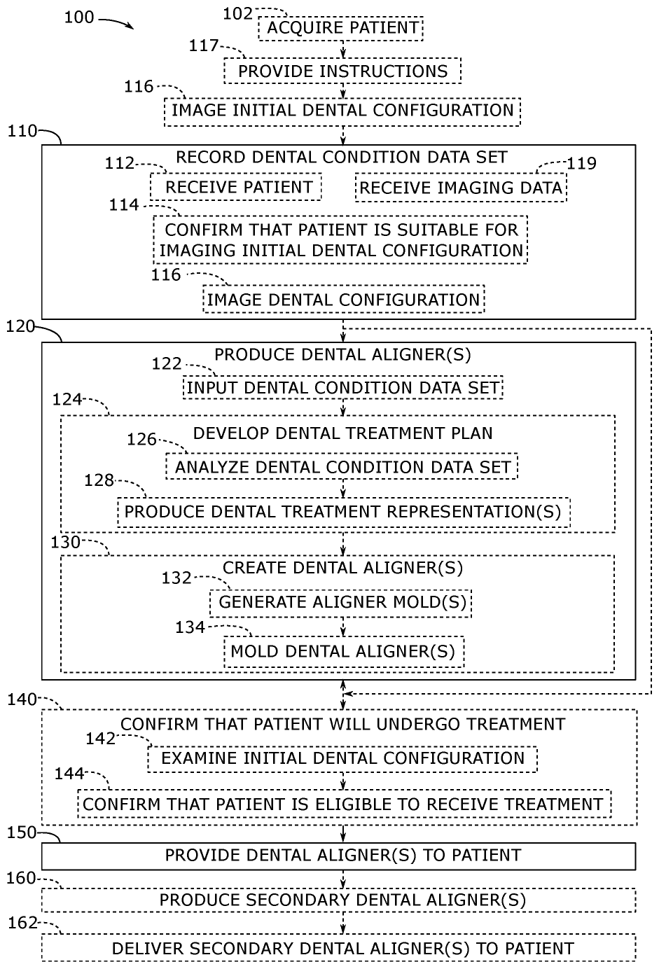

### RELATED APPLICATIONS

This application claims the benefit of U.S. Provisional patent application Ser. Nos. 63/184,374 and 63/185,806, filed on May 5, 2021 and May 7, 2021, respectively, and the complete disclosures of which are incorporated herein by reference.

### FIELD

The present disclosure is directed generally to the orthodontic field, and more particularly to methods of initiating aligner-based orthodontic treatment of a patient.

### BACKGROUND

Orthodontic treatment to modify and/or correct the configuration of a patient's teeth may be performed in any of a variety of manners. While some traditional forms of orthodontic treatment involve exerting forces on the patient's teeth with wires retained in tooth-mounted brackets, such orthodontic wires and brackets may be uncomfortable to the patient, and they generally require in-office adjustment by a skilled orthodontist. More recent forms of orthodontic treatment utilize dental aligners that are removably worn by the patient and that exert forces on the patient's teeth to transition the patient's teeth toward a configuration defined by the aligners. As examples, dental aligners may be utilized to treat many instances of malocclusion and/or dental misalignment that previously were treated with tooth-mounted orthodontic brackets. Such dental aligners generally must be custom-fit to the patient, which may cause undesirable delays in the initiation of the orthodontic treatment. Thus, there exists a need for improved methods of initiating aligner-based orthodontic treatment of a patient.

### SUMMARY

Methods of initiating aligner-based orthodontic treatment of a patient are disclosed herein. A method of initiating aligner-based orthodontic treatment of a patient includes recording a dental condition data set that at least partially characterizes an initial dental configuration of at least a subset of the patient's teeth, producing one or more dental aligners, and providing the one or more dental aligners to the patient. The producing the one or more dental aligners is performed subsequent to the recording the dental condition data set, and the providing the one or more dental aligners to the patient is performed subsequent to the producing the one or more dental aligners.

### BRIEF DESCRIPTION OF THE DRAWING

The FIGURE is a flowchart representing examples of methods, according to the present disclosure, of initiating aligner-based orthodontic treatment of a patient.

### DESCRIPTION

The FIGURE is a flowchart depicting examples of methods 100 of initiating aligner-based orthodontic treatment of a patient. In the FIGURE, some steps are illustrated in dashed boxes indicating that such steps may be optional or may correspond to an optional version of a method according to the present disclosure. That said, not all methods according to the present disclosure are required to include the steps illustrated in solid boxes. The methods and steps illustrated in the FIGURE are not limiting and other methods and steps are within the scope of the present disclosure, including methods having greater than or fewer than the number of steps illustrated, as understood from the discussions herein.

As shown in the FIGURE, a method 100 of initiating aligner-based orthodontic treatment of a patient includes recording, at 110, a dental condition data set; producing, at 120, one or more dental aligners; and providing, at 150, the one or more dental aligners to the patient. In particular, the dental condition data set at least partially characterizes an initial dental configuration of at least a subset of the patient's teeth. For example, the dental condition data set may characterize and/or represent an orientation, an alignment, a bite pattern, etc. that characterizes one or more of the patient's teeth and which may characterize a subset or all of the teeth in the patient's upper and/or lower jaw. The dental condition data set thus may indicate and/or represent a misalignment, a malocclusion, and/or other dental condition to be treated via aligner-based orthodontic treatment. Accordingly, the one or more dental aligners that are produced and provided to the patient in the course of methods 100 may be configured to alleviate, treat, and/or remedy the dental condition that is represented by the dental condition data set.

The producing the dental aligner(s) at 120 is performed subsequent to the recording the dental condition data set at 110, and the providing the one or more dental aligners to the patient at 150 is performed subsequent to the producing the dental aligner(s) at 120. Additionally, each of the recording the dental condition data set at 110, the producing the dental aligner(s) at 120, and the providing the one or more dental aligners to the patient at 150 may be performed at a service provider office while the patient remains at the service provider office. In this manner, methods 100 according to the present disclosure stand in contrast with prior methods of initiating aligner-based orthodontic treatment of a patient, in which the patient typically must wait until long after leaving the office of the service provider, typically at least a week or several weeks, to receive dental aligners. Because the recording the dental condition data set at 110, the producing the dental aligner(s) at 120, and the providing the one or more dental aligners to the patient at 150 may be performed while the patient remains at the service provider office, the patient thus is able to begin the aligner-based orthodontic treatment immediately. As more specific examples, the recording the dental condition data set at 110 and the producing the dental aligner(s) at 120 may be performed within a total time span that is at most 3 hours, at most 2 hours, at most 90 minutes, and/or at most 1 hour. In some examples, the providing the one or more dental aligners to the patient at 150 occurs the same day or the next day as the recording the dental condition data set at 110 and the producing the dental aligner(s) at 120, with examples including within at most 24 hours, at most 18 hours, at most 12 hours, at most 6 hours, at most 3 hours, at most 2 hours, at most 90 minutes, and/or at most 1 hour. In some examples, the recording the dental condition data set at 110, the producing the dental aligner(s) at 120, and the providing the one or more dental aligners to the patient at 150 may be performed within a total time span that is at most 3 hours, at most 2 hours, at most 90 minutes, at most 1 hour, and/or within the same day.

In some examples, however, one or more aspects of the recording the dental condition data set at 110 may be performed outside of a service provider office and/or prior to the patient's arrival at the service provider office. For example, the recording the dental set at 110 may be performed prior to the patient's arrival at the service provider office.

As used herein, the term “service provider” may refer to any individual, or collection of individuals, that performs one or more steps of methods 100 according to the present disclosure. As examples, the term “service provider” may refer to a doctor, an orthodontist, a dentist, an orthodontic and/or dental assistant, an orthodontic and/or dental technician, an orthodontic and/or dental office staff member, etc. Similarly, references herein to a “service provider office” may be understood as referring to any suitable physical location and/or place of business at which the service provider operates, such as an orthodontist office or dentist office that may be visited by the patient to initiate the aligner-based orthodontic treatment.

The recording the dental condition data set at 110 may include any of a variety of steps for evaluating and/or characterizing the initial dental configuration of the patient's teeth. As used herein, the phrase “initial dental configuration,” as used to describe and/or characterize the patient's teeth, is intended to refer to the dental configuration of the patient's teeth at the time that the recording the dental condition data set at 110 is performed. Additionally, as used herein, the term “dental configuration” is intended to refer to any of a variety of characteristics and/or conditions of the patient's teeth, such as an alignment of the patient's teeth, a bite pattern of the patient's teeth, a malocclusion of the patient's teeth, etc. The term “dental configuration” additionally may be understood as referring to any suitable subset of the patient's teeth, such as one or more teeth (and optionally most or all) of the patient's upper jaw and/or one or more teeth (and optionally most or all) of the patient's lower jaw.

In some examples, and as shown in the FIGURE, the recording the dental condition data set at 110 includes receiving, at 112, the patient at the service provider office and imaging, at 116, the initial dental configuration. The imaging the initial dental configuration at 116 may include imaging in one or more of any of a variety of manners. As an example, the imaging the initial dental configuration at 116 may include recording one or more X-ray images of the initial dental configuration, such that the dental condition data set includes the one or more X-ray images. Additionally or alternatively, the imaging the initial dental configuration at 116 may include recording one or more photographs of the initial dental configuration, such that the dental condition data set includes the one or more photographs. Additionally or alternatively, the imaging the initial dental configuration at 116 may include performing a three-dimensional scan of the initial dental configuration, such that the dental condition data set includes data that is produced by the three-dimensional scan. As more specific examples, the performing the three-dimensional scan of the initial dental configuration may include performing a dental computed tomography (CT) scan and/or a cone beam computed tomography (CBCT) scan.

In some examples, and as additionally shown in the FIGURE, the recording the dental condition data set at 110 further may include, prior to the imaging the initial dental configuration at 116, confirming, at 114, that the patient is suitable for imaging the initial dental configuration. For example, the confirming that the patient is suitable at 114 may include receiving information from the patient, such as via a written questionnaire and/or via verbal questioning, to identify any reasons that the patient may be ill-suited to undergo the imaging the initial dental configuration at 116. As more specific examples, the confirming that the patient is suitable at 114 may include receiving information from the patient regarding whether the patient has a medical condition, such as epilepsy, whether the patient is pregnant, whether the patient is able to lean back in an examination chair, etc. Accordingly, in such examples, the imaging the initial dental configuration at 116 and/or one or more subsequent steps of methods 100 may be performed only if the confirming that the patient is suitable at 114 results in a determination that the patient is suitable for imaging the initial dental configuration.

In some examples and optionally illustrated in the FIGURE, the imaging the initial dental configuration at 116 may take place at least partially prior to the recording the dental condition data set at 110. For example, the imaging the initial dental configuration at 116 may include the patient, a family member of the patient, and/or another third party imaging the patient's initial dental configuration outside of the service provider office, such as at home or at a referring provider's office, such as a dentist or other service provider. In some such examples, as illustrated in the FIGURE, a method 100 of initiating aligner-based orthodontic treatment of a patient may further include providing, at 117, the patient with instructions and/or the necessary device(s) (e.g., imaging hardware, instrument(s), software, guide(s), etc.) to perform the imaging the initial dental configuration at 116.

Accordingly, the initial dental configuration and/or the dental condition data set, in some examples, may include and/or be based at least in part on information obtained from one or more of the patient, a third party, and the service provider. For example, the initial dental configuration and/or the dental condition data set may be based in part on photographs taken by the patient or a third party, and on X-ray imaging and/or three-dimensional scanning performed by the service provider at the service provider's office.

In some examples, the recording the dental condition data set at 110 therefore includes receiving, at 119, imaging data (e.g., photographs), or at least a portion of the initial dental configuration, from the patient or third party that initially acquired the initial dental configuration or portion thereof, such as via the optional imaging the initial dental configuration at 116 prior to the recording at 110. In some such examples, the imaging data is received prior to the patient's arrival at the service provider's office, such as via email or file transfer.

The producing the dental aligner(s) at 120 may include utilizing the dental configuration data set produced via the recording the dental condition data set at 110 in any of a variety of manners. For example, and as shown in the FIGURE, the producing the dental aligner(s) at 120 may include inputting, at 122, the dental condition data set into a computer that is programmed to run, or execute, a dental analysis program. In such examples, the producing the dental aligner(s) at 120 additionally may include developing, at 124, a dental treatment plan. In particular, in such examples, the developing the dental treatment plan is at least partially performed by the computer utilizing the dental analysis program and at least partially based upon the dental condition data set.

In such examples, the dental analysis program may include and/or be any of a variety of computerized routines for developing at least a portion of the dental treatment plan. For example, the dental treatment plan may include and/or be a plan (e.g., a sequence of steps, treatments, dental aligners, etc.) for transitioning the initial dental configuration of the patient to a final dental configuration of the patient, and the developing the dental treatment plan at 124 may include modeling one or more dental aligners to produce the final dental configuration. More specifically, when the developing the dental treatment plan at 124 includes utilizing a computer-based dental analysis program, one or more steps and/or features of the treatment plan may be generated automatically by the computer and/or without human input. In such examples, the developing the dental treatment plan at 124 may be described as being performed at least partially automatically, and/or as being at least partially performed by an automated program. Additionally or alternatively, in some examples, the developing the dental treatment plan at 124 may include configuring, supplementing, and/or customizing one or more aspects of the dental analysis program, such as via human input and/or intervention.

In some examples, the dental analysis program may utilize artificial intelligence and/or may receive data based on machine learning algorithms to optimize the dental treatment plan. For example, the received data may be associated with or based on prior patient data acquired by the service provider and/or by third parties that perform methods 100 or similar methods thereto. For example, a party may maintain a database of patient orthodontic records, including initial dental configurations, dental condition data sets, dental treatment plans, dental treatment representations, representations of dental aligners, representations of aligner molds, and so forth, with the machine learning algorithms being used on such data to determine optimized dental treatment plans.

As used herein, the term “final dental configuration” may describe and/or correspond to a dental configuration that alleviates and/or mitigates one or more issues associated with the initial dental configuration, such as by improving an alignment, a positioning, and/or an orientation of one or more of the patient's teeth. In some examples, the final dental configuration may include and/or be a predetermined dental configuration, and/or may include and/or be a dental configuration that is customized and/or tailored to the patient.

The inputting the dental condition data set at 122 may be performed in any of a variety of manners, such as by electronically transferring the dental condition data set to the computer via a wired connection, a wireless connection, a physical storage medium, and/or a portable physical storage medium. Additionally or alternatively, the inputting the dental condition data set at 122 may include manually inputting at least a portion of the dental condition data set to the computer. Inputting the dental condition set at 122 also may include accessing, or permitting or providing access to, the dental condition set by the computer.

In examples in which the producing the dental aligner(s) at 120 includes utilizing a computerized dental analysis program, the dental analysis program may utilize the dental condition data set in any of a variety of manners. For example, and as shown in the FIGURE, the developing the dental treatment plan at 124 may include analyzing, at 126, the dental condition data set to produce an initial dental representation and producing, at 128, one or more dental treatment representations. The analyzing at 126 and/or the producing at 128 may be performed with and/or by the dental analysis program, and may be based at least in part on inputs and decisions by the service provider. In some examples, the producing the dental treatment representation(s) at 128 is at least partially based on the analyzing the dental condition data set at 126.

When performed, the analyzing the dental condition data set at 126 includes producing an initial dental representation that represents the initial dental configuration of the patient. In such examples, the initial dental representation may include and/or be a representation, such as a virtual and/or computer-based representation, of the initial dental configuration in a format that is suitable to be utilized and/or manipulated by the dental analysis program. As a more specific example, the dental condition data set may include one or more X-ray images characterizing the initial dental condition, and the analyzing the dental condition data set at 126 may include producing an initial dental representation that includes a geometrical representation of the X-ray image(s). Additionally or alternatively, in some examples, such as when the dental condition data set includes data that is produced by a three-dimensional scan, the initial dental representation may include and/or be at least a portion of the dental condition data set itself. In some examples, the initial dental representation and the dental treatment representation(s) each includes, or is, a three-dimensional representation of one or more of the patient's teeth.

When performed, the producing the dental treatment representation(s) at 128 includes producing such that each dental treatment representation represents a respective target dental configuration. As used herein, the term “target dental configuration” is intended to refer to a dental configuration that a given (e.g., a particular) dental aligner is configured to produce in the patient's teeth. In particular, transitioning the patient's teeth from the initial dental configuration to the final dental configuration may include utilizing a sequence of a plurality of dental aligners, each of which is configured to transition the dental configuration of the patient incrementally toward, or to, the final dental configuration. Stated differently, the producing the dental treatment representation(s) at 128 may include producing such that the initial dental representation and the one or more dental treatment representations collectively represent a sequential progression of dental configurations from the initial dental configuration to the final dental configuration. In this manner, the one or more target dental configurations may include the final dental configuration.

In some examples, and as shown in the FIGURE, methods 100 further include, subsequent to the developing the dental treatment plan at 124, creating, at 130, the one or more dental aligner(s). In such examples, the creating the dental aligner(s) at 130 may be performed in any of a variety of manners. For example, and as shown in the FIGURE, the creating the dental aligner(s) at 130 may include, for each respective dental aligner, generating, at 132, a respective aligner mold that represents one of the target dental configurations and molding, at 134 and with the respective aligner mold, the respective dental aligner. Stated differently, for each target dental configuration produced in the producing the dental treatment representation(s) at 128, the respective dental aligner corresponding to the target dental configuration may be produced by creating the respective aligner mold corresponding to the respective dental aligner and utilizing the respective aligner mold to form the respective dental aligner. In such examples, the generating the respective aligner mold at 132 may be performed in any suitable manner, such as via an additive manufacturing process and/or a subtractive manufacturing process. Additionally, in such examples, the molding the respective dental aligner at 134 may be performed in any suitable manner, such as by applying an aligner material to the respective aligner mold and curing the aligner material to produce the respective dental aligner. The creating the dental aligner(s) at 130 additionally or alternatively may include forming the aligners directly from the dental treatment representations produced at 128 without creating aligner molds. For example, the aligners may be printed by an additive manufacturing process, such as 3D printing, or a subtractive manufacturing process, such as milling, directly from the dental treatment representations.

In some examples, and as shown in the FIGURE, methods 100 additionally include, prior to the providing the dental aligner(s) to the patient at 150, confirming, at 140, that the patient will undergo treatment. In particular, in some such examples, and as shown in the FIGURE, the confirming that the patient will undergo treatment at 140 includes examining, at 142, the initial dental configuration and/or confirming, at 144, that the patient is eligible to receive aligner-based orthodontic treatment. In such examples, each of the examining the initial dental configuration at 142 and the confirming that the patient is eligible at 144 is performed by a service provider, such as a doctor, a dentist, an orthodontist, and/or an assistant thereof.

When performed, the examining the initial dental configuration at 142 may be (but is not required in all examples to be) performed by an individual that is different than an individual that performs at least a portion of the recording the dental condition data set at 110. For example, the recording the dental condition data set at 110 may be at least partially performed by an assistant and/or technician, while the examining the initial dental configuration at 142 may be performed by a doctor, a dentist, and/or an orthodontist. In this manner, the role of the dentist and/or orthodontist in performing aspects of methods 100 may be limited to a subset of the steps of methods 100, such that two or more steps of methods 100 may be performed at least partially concurrently. As more specific examples, the examining the initial dental configuration at 142 may be performed subsequent to the recording the dental condition data set at 110, and/or may be performed at least partially concurrently with one or more other steps of the producing the dental aligner(s) at 120. Such a sequence thus may enable methods 100 to proceed from the recording the dental condition data set at 110 to the providing the dental aligner(s) to the patient at 150 in a short amount of time relative to a method in which initiating production of the dental aligners is contingent upon examination of the initial dental configuration by the service provider. Stated differently, performing the recording the dental condition data set at 110 prior to the examining the initial dental configuration at 142 enables the producing the dental aligner(s) at 120 to be initiated prior to, and/or at least partially concurrent with, the confirming that the patient will undergo treatment at 140. As a result, the patient need not wait for the dental aligners to be produced only subsequent to the examining the initial dental configuration at 142. However, it also is within the scope of the present disclosure that one or more steps of the producing the dental aligner(s) at 120 may be performed subsequent to, and/or responsive to, the examining the initial dental configuration at 142. For example, the creating the dental aligner(s) at 130 may be performed subsequent to the examining the initial dental configuration at 142. Additionally or alternatively, the creating the dental aligner(s) at 130 and/or the providing the dental aligner(s) to the patient at 150 may be performed only subsequent to the confirming that the patient will undergo treatment at 140 and/or the confirming that the patient is eligible at 144.

When performed, the examining the initial dental configuration at 142 may include performing any suitable examination. For example, the examining the initial dental configuration at 142 may include visually and/or manually inspecting the patient's teeth to confirm the initial dental configuration as determined in the recording the dental condition data set at 110 and/or the dental treatment plan as developed in the developing the dental treatment plan at 124. Similarly, when performed, the confirming that the patient is eligible at 144 may pertain to any of a variety of considerations. For example, the confirming that the patient is eligible at 144 may include confirming that the patient is medically fit to undergo aligner-based orthodontic treatment. In particular, as used herein, the term “eligible,” as used to describe a fitness of the patient to undergo aligner-based orthodontic treatment, is intended to describe a condition in which the patient is not subject to any conditions and/or considerations that would preclude the patient from receiving and/or benefiting from the aligner-based orthodontic treatment.

In some examples, the confirming that the patient will undergo treatment at 140 additionally or alternatively includes one or more steps for formalizing a relationship between the patient and the service provider. For example, the confirming that the patient will undergo treatment at 140 may include confirming payment, or a payment plan, for the aligner-based orthodontic treatment and/or formally admitting the patient as a patient of the service provider.

The producing the dental aligner(s) at 120 may include producing any suitable number (e.g., quantity) of dental aligners. As examples, the producing the dental aligner(s) at 120 may include producing one dental aligner, two dental aligners, three dental aligners, four dental aligners, or more than four dental aligners. The dental aligners provided during the producing the dental aligner(s) at 120 may include a series of dental aligners for sequential use to treat the teeth in the patient's upper and/or lower jaw.

In some examples, the producing the dental aligner(s) at 120 includes creating at least a primary dental aligner, such that the providing the dental aligner(s) to the patient at 150 includes providing at least the primary dental aligner. Additionally or alternatively, in some examples, methods 100 may include providing the patient with one or more supplemental dental aligners after the patient has departed the service provider office. For example, and as shown in the FIGURE, methods 100 further may include producing, at 160, at least one secondary dental aligner that is configured to be utilized by the patient subsequent to utilizing the primary dental aligner. In such examples, methods 100 further may include, subsequent to the patient departing the service provider office, delivering, at 162, the at least one secondary dental aligner to the patient. In this manner, the producing the secondary dental aligner(s) at 160 and/or the delivering the secondary dental aligner(s) to the patient at 162 may be described as a continuation of the aligner-based orthodontic treatment that is initiated by the recording the dental condition data set at 110, the producing the dental aligner(s) at 120, and the providing the dental aligner(s) to the patient at 150.

In some examples, and as shown in the FIGURE, methods 100 additionally may include, prior to the recording the dental condition data set at 110, acquiring, at 102, the patient. In such examples, the acquiring the patient at 102 may include any of a variety of steps for soliciting the patient and/or making the patient aware of the services of the service provider. As more specific examples, the acquiring the patient at 102 may include advertising one or more aspects of methods 100 of initiating aligner-based orthodontic treatment, receiving a referral from a third party, such as a dentist, scheduling an appointment for the patient to visit the service provider office, and/or receiving the patient at the service provider office without an appointment. Stated differently, in various examples, methods 100 may be performed with respect to a patient that previously has made an appointment to visit the service provider office, or may be performed with respect to a “walk-in” patient who is aware of the services of the service provider but has not previously made plans to visit the service provider office. In this manner, methods 100 may facilitate the rapid initiation of aligner-based orthodontic treatment for a wide variety of patients.

Illustrative, non-exclusive examples of inventive subject matter according to the present disclosure are described in the following enumerated paragraphs:

A1. A method of initiating aligner-based orthodontic treatment of a patient, the method comprising:

recording a dental condition data set that at least partially characterizes an initial dental configuration of at least a subset of the patient's teeth;

subsequent to the recording the dental condition data set, producing one or more dental aligners; and

subsequent to the producing the one or more dental aligners, providing the one or more dental aligners to the patient.

A2. The method of paragraph A1, wherein the producing the one or more dental aligners, and the providing the one or more dental aligners to the patient are performed at a service provider office while the patient remains at the service provider office.

A3. The method of paragraph A2, wherein the recording the dental condition data set also is performed at the service provider office while the patient remains at the service provider office.

A4. The method of any of paragraphs A1-A3, wherein the producing the one or more dental aligners and the providing the one or more dental aligners to the patient are performed within a total time span that is one or more of at most 3 hours, at most 2 hours, at most 90 minutes, and/or at most 1 hour.

A5. The method of paragraph A4, wherein the recording the dental condition data set also is performed within the total time span.

A6. The method of any of paragraphs A1-A5, wherein the one or more dental aligners includes one of one dental aligner, two dental aligners, three dental aligners, four dental aligners, or more than four dental aligners.

A7. The method of any of paragraphs A1-A6, further comprising imaging the initial dental configuration.

A8. The method of paragraph A7, wherein the imaging the initial dental configuration is performed at least in part, and optionally fully, at a/the service provider office while the patient remains at the service provider office.

A9. The method of any of paragraphs A7-A8, wherein the imaging the initial dental configuration is performed by the patient or a third party away from a/the service provider office prior to the patient arriving at the service provider office.

A10. The method of any of paragraphs A1-A9, wherein the recording the dental condition data set includes receiving imaging data associated with the initial dental configuration, optionally wherein the imaging data is, or was, provided by the patient or a third party.

A11. The method of any of paragraphs A1-A10, wherein the recording the dental condition data set includes receiving the patient at a/the service provider office.

A12. The method of paragraph A11, wherein the recording the dental condition data set further comprises an/the imaging the initial dental configuration to produce the dental condition data set.

A13. The method of any of paragraphs A7-A12, wherein the recording the dental condition data set includes, prior to the imaging the initial dental configuration, confirming that the patient is suitable for imaging the initial dental configuration.

A14. The method of paragraph A13, wherein the confirming that the patient is suitable for imaging the initial dental configuration includes receiving information from the patient regarding one or more of:

(i) whether the patient has a medical condition, optionally epilepsy;

(ii) whether the patient is pregnant; and

(iii) whether the patient is able to lean back in an examination chair.

A15. The method of any of paragraphs A7-A14, wherein the imaging the initial dental configuration includes recording one or more X-ray images of the initial dental configuration; and wherein the dental condition data set includes the one or more X-ray images.

A16. The method of any of paragraphs A7-A15, wherein the imaging the initial dental configuration includes recording one or more photographs of the initial dental configuration; and wherein the dental condition data set includes the one or more photographs.

A17. The method of any of paragraphs A7-A16, wherein the imaging the initial dental configuration includes performing a three-dimensional scan of the initial dental configuration; and wherein the dental condition data set includes data produced by the three-dimensional scan.

A18. The method of paragraph A17, wherein the performing the three-dimensional scan of the initial dental configuration includes performing one or both of a dental computed tomography (CT) scan and a cone beam computed tomography (CBCT) scan.

A19. The method of any of paragraphs A1-A18, wherein the producing the one or more dental aligners includes:

inputting the dental condition data set into a computer that is programmed to run a dental analysis program; and

developing a dental treatment plan;

wherein the developing the dental treatment plan is performed, at least in part, by the computer utilizing the dental analysis program and based, at least in part, on the dental condition data set.

A20. The method of paragraph A19, wherein the inputting the dental condition data set includes electronically transferring the dental condition data set to the computer, optionally via one or more of a wired connection, a wireless connection, a physical storage medium, and a portable physical storage medium.

A21. The method of any of paragraphs A19-A20, wherein the inputting the dental condition data set includes manually inputting at least a portion of the dental condition data set to the computer.

A22. The method of any of paragraphs A19-A21, wherein the developing the dental treatment plan includes:

analyzing, with the dental analysis program, the dental condition data set to produce an initial dental representation that represents the initial dental configuration of the patient; and

producing, with the dental analysis program, one or more dental treatment representations that represent a respective one or more target dental configurations.

A23. The method of paragraph A22, wherein the producing the one or more dental treatment representations is based, at least in part, on the analyzing the dental condition data set.

A24. The method of any of paragraphs A22-A23, wherein the producing the one or more dental treatment representations includes producing such that the initial dental representation and the one or more dental treatment representations collectively represent a sequential progression of dental configurations from the initial dental configuration of the patient to a final dental configuration of the patient.

A25. The method of any of paragraphs A22-A24, wherein each of the initial dental representation and the one or more dental treatment representations includes and/or is a three-dimensional representation of one or more teeth of the patient.

A26. The method of any of paragraphs A19-A25, wherein the producing the one or more dental aligners further includes, subsequent to the developing the dental treatment plan, creating the one or more dental aligners.

A27. The method of paragraph A26, wherein the creating the one or more dental aligners includes, for each respective dental aligner of the one or more dental aligners:

generating a respective aligner mold that represents one of a/the one or more target dental configurations; and

molding, with the respective aligner mold, the respective dental aligner.

A28. The method of paragraph A27, wherein the generating the respective aligner mold includes generating via at least one of an additive manufacturing process and a subtractive manufacturing process.

A29. The method of any of paragraphs A27-A28, wherein the molding the respective dental aligner includes:

applying an aligner material to the respective aligner mold; and

curing the aligner material.

A30. The method of paragraph A26, wherein the creating the one or more dental aligners includes, for each respective dental aligner of the one or more dental aligners, generating the respective dental aligner via an additive manufacturing process.

A31. The method of any of paragraphs A1-A30, wherein the producing the one or more dental aligners includes creating at least a primary dental aligner; and wherein the providing the one or more dental aligners to the patient includes providing at least the primary dental aligner.

A32. The method of paragraph A31, further comprising:

creating at least one secondary dental aligner that is configured to be utilized by the patient subsequent to utilizing the primary dental aligner; and

subsequent to the patient departing a/the service provider office, delivering the at least one secondary dental aligner to the patient.

A33. The method of any of paragraphs A1-A32, further comprising, prior to the providing the one or more dental aligners to the patient, confirming that the patient will undergo treatment.

A34. The method of paragraph A33, wherein the confirming that the patient will undergo treatment includes examining the initial dental configuration of the patient; wherein the examining the initial dental configuration is performed by a service provider.

A35. The method of paragraph A34, wherein the examining the initial dental configuration is performed subsequent to the recording the dental condition data set.

A36. The method of any of paragraphs A34-A35, wherein the service provider is one or more of a doctor, a dentist, and an orthodontist.

A37. The method of any of paragraphs A34-A36, wherein the examining the initial dental configuration includes confirming, by the service provider, that the patient is eligible to receive aligner-based orthodontic treatment.

A38. The method of paragraph A37, wherein the confirming that the patient is eligible to receive aligner-based orthodontic treatment includes confirming that the patient is medically fit to undergo aligner-based orthodontic treatment.

A39. The method of any of paragraphs A34-A38, wherein the examining the initial dental configuration and the producing the one or more dental aligners are performed at least partially concurrently.

A40. The method of any of paragraphs A34-A39, wherein a/the creating the one or more dental aligners is performed subsequent to the examining the initial dental configuration.

A41. The method of any of paragraphs A33-A40, wherein one or both of a/the creating the one or more dental aligners and the providing the one or more dental aligners to the patient is performed only subsequent to one or both of the confirming that the patient will undergo treatment and a/the confirming that the patient is eligible to receive aligner-based orthodontic treatment.

A42. The method of any of paragraphs A33-A41, wherein the confirming that the patient will undergo treatment includes confirming payment for the aligner-based orthodontic treatment.

A43. The method of any of paragraphs A33-A42, wherein the confirming that the patient will undergo treatment includes formally admitting the patient as a patient of a/the service provider.

A44. The method of any of paragraphs A1-A43, further comprising, prior to the recording the dental condition data set, acquiring the patient.

A45. The method of paragraph A44, wherein the acquiring the patient includes one or more of:

(i) receiving a referral from a third party;

(ii) advertising one or more aspects of the method of initiating aligner-based orthodontic treatment;

(iii) scheduling an appointment for the patient to visit a/the service provider office; and

(iv) receiving the patient at the service provider office without an appointment.

In the present disclosure, several of the illustrative, non-exclusive examples have been discussed and/or presented in the context of flow diagrams, or flow charts, in which the methods are shown and described as a series of blocks, or steps. Unless specifically set forth in the accompanying description, it is within the scope of the present disclosure that the order of the blocks may vary from the illustrated order in the flow diagram, including with two or more of the blocks (or steps) occurring in a different order, concurrently, and/or repeatedly. It is also within the scope of the present disclosure that the blocks, or steps, may be implemented as logic, which also may be described as implementing the blocks, or steps, as logics. In some applications, the blocks, or steps, may represent expressions and/or actions to be performed by functionally equivalent circuits or other logic devices. The illustrated blocks may, but are not required to, represent executable instructions that cause a computer, processor, and/or other logic device to respond, to perform an action, to change states, to generate an output or display, and/or to make decisions.

As used herein, the terms “adapted” and “configured” mean that the element, component, or other subject matter is designed and/or intended to perform a given function. Thus, the use of the terms “adapted” and “configured” should not be construed to mean that a given element, component, or other subject matter is simply “capable of” performing a given function but that the element, component, and/or other subject matter is specifically selected, created, implemented, utilized, programmed, and/or designed for the purpose of performing the function. It is also within the scope of the present disclosure that elements, components, and/or other recited subject matter that is recited as being adapted to perform a particular function may additionally or alternatively be described as being configured to perform that function, and vice versa. Similarly, subject matter that is recited as being configured to perform a particular function may additionally or alternatively be described as being operative to perform that function.

As used herein, the term “and/or” placed between a first entity and a second entity means one of (1) the first entity, (2) the second entity, and (3) the first entity and the second entity. Multiple entries listed with “and/or” should be construed in the same manner, i.e., “one or more” of the entities so conjoined. Other entities optionally may be present other than the entities specifically identified by the “and/or” clause, whether related or unrelated to those entities specifically identified. Thus, as a non-limiting example, a reference to “A and/or B,” when used in conjunction with open-ended language such as “comprising,” may refer, in one example, to A only (optionally including entities other than B); in another example, to B only (optionally including entities other than A); in yet another example, to both A and B (optionally including other entities). These entities may refer to elements, actions, structures, steps, operations, values, and the like.

As used herein, the phrase “at least one,” in reference to a list of one or more entities should be understood to mean at least one entity selected from any one or more of the entities in the list of entities, but not necessarily including at least one of each and every entity specifically listed within the list of entities and not excluding any combinations of entities in the list of entities. This definition also allows that entities may optionally be present other than the entities specifically identified within the list of entities to which the phrase “at least one” refers, whether related or unrelated to those entities specifically identified. Thus, as a non-limiting example, “at least one of A and B” (or, equivalently, “at least one of A or B,” or, equivalently “at least one of A and/or B”) may refer, in one embodiment, to at least one, optionally including more than one, A, with no B present (and optionally including entities other than B); in another embodiment, to at least one, optionally including more than one, B, with no A present (and optionally including entities other than A); in yet another embodiment, to at least one, optionally including more than one, A, and at least one, optionally including more than one, B (and optionally including other entities). In other words, the phrases “at least one,” “one or more,” and “and/or” are open-ended expressions that are both conjunctive and disjunctive in operation. For example, each of the expressions “at least one of A, B, and C,” “at least one of A, B, or C,” “one or more of A, B, and C,” “one or more of A, B, or C,” and “A, B, and/or C” may mean A alone, B alone, C alone, A and B together, A and C together, B and C together, A, B, and C together, and optionally any of the above in combination with at least one other entity.

As used herein, the phrase “at least substantially,” when modifying a degree or relationship, includes not only the recited “substantial” degree or relationship, but also the full extent of the recited degree or relationship. A substantial amount of a recited degree or relationship may include at least 75% of the recited degree or relationship. For example, a first component that extends at least substantially around a second component includes a first component that extends around at least 75% of a circumference of the second component and also includes a first component that extends fully circumferentially around the second component.

As used herein, the phrase, “for example,” the phrase, “as an example,” and/or simply the term “example,” when used with reference to one or more components, features, details, structures, embodiments, and/or methods according to the present disclosure, are intended to convey that the described component, feature, detail, structure, embodiment, and/or method is an illustrative, non-exclusive example of components, features, details, structures, embodiments, and/or methods according to the present disclosure. Thus, the described component, feature, detail, structure, embodiment, and/or method is not intended to be limiting, required, or exclusive/exhaustive; and other components, features, details, structures, embodiments, and/or methods, including structurally and/or functionally similar and/or equivalent components, features, details, structures, embodiments, and/or methods, are also within the scope of the present disclosure.

A computer may be any suitable device or devices that are configured to perform the functions of the computer discussed herein. For example, the computer may include one or more of an electronic computer, a dedicated computer, a special-purpose computer, a personal computer, a display device, a logic device, a memory device, and/or a memory device having non-transitory computer-readable media suitable for storing computer-executable instructions for implementing aspects of systems and/or methods according to the present disclosure.

Additionally or alternatively, the computer may include, or be configured to read, non-transitory computer-readable storage, or memory, media suitable for storing computer-executable instructions, or software, for implementing methods or steps of methods according to the present disclosure. Examples of such media include CD-ROMs, disks, hard drives, flash memory, etc. As used herein, storage, or memory, devices and media having computer-executable instructions as well as computer-implemented methods and other methods according to the present disclosure are considered to be within the scope of subject matter deemed patentable in accordance with Section 101 of Title 35 of the United States Code.

The various disclosed elements of methods disclosed herein are not required to all methods according to the present disclosure, and the present disclosure includes all novel and non-obvious combinations and subcombinations of the various methods disclosed herein. Moreover, one or more of the various steps and/or aspects disclosed herein may define independent inventive subject matter that is separate and apart from the whole of the disclosed methods. Accordingly, such inventive subject matter is not required to be associated with the specific methods that are expressly disclosed herein, and such inventive subject matter may find utility in methods that are not expressly disclosed herein.

### INDUSTRIAL APPLICABILITY

The methods disclosed herein are applicable to the dental and orthodontics industries.

It is believed that the disclosure set forth above encompasses multiple distinct inventions with independent utility. While each of these inventions has been disclosed in its preferred form, the specific embodiments thereof as disclosed and illustrated herein are not to be considered in a limiting sense as numerous variations are possible. The subject matter of the inventions includes all novel and non-obvious combinations and subcombinations of the various elements, features, functions and/or properties disclosed herein. Similarly, when the disclosure or subsequently filed claims recite “a” or “a first” element or the equivalent thereof, such disclosure and/or claims should be understood to include incorporation of one or more such elements, neither requiring nor excluding two or more such elements.

It is believed that the following claims particularly point out certain combinations and subcombinations that are directed to one of the disclosed inventions and are novel and non-obvious. Inventions embodied in other combinations and subcombinations of features, functions, elements, and/or properties may be claimed through amendment of the present claims or presentation of new claims in this or a related application. Such amended or new claims, whether they are directed to a different invention or directed to the same invention, whether different, broader, narrower, or equal in scope to the original claims, are also regarded as included within the subject matter of the inventions of the present disclosure.

1. A method of initiating aligner-based orthodontic treatment of a patient, the method comprising:
recording a dental condition data set that at least partially characterizes an initial dental configuration of at least a subset of the patient's teeth;
subsequent to the recording the dental condition data set, producing one or more dental aligners; and
subsequent to the producing the one or more dental aligners, providing the one or more dental aligners to the patient;
wherein the producing the one or more dental aligners and the providing the one or more dental aligners to the patient are performed at a service provider office while the patient remains at the service provider office.

2. The method of claim 1, wherein the recording the dental condition data set also is performed at the service provider office while the patient remains at the service provider office.

3. The method of claim 1, wherein the producing the one or more dental aligners and the providing the one or more dental aligners to the patient are performed within a total time span that is at most 3 hours.

4. The method of claim 3, wherein the recording the dental condition data set also is performed within the total time span.

5. The method of claim 1, further comprising imaging the initial dental configuration, wherein the imaging the initial dental configuration is performed at the service provider office while the patient remains at the service provider office.

6. The method of claim 5, wherein the recording the dental condition data set includes, prior to the imaging the initial dental configuration, confirming that the patient is suitable for imaging the initial dental configuration.

7. The method of claim 5, wherein one or more of:
the imaging the initial dental configuration includes recording one or more X-ray images of the initial dental configuration, wherein the dental condition data set includes the one or more X-ray images;
the imaging the initial dental configuration includes recording one or more photographs of the initial dental configuration, wherein the dental condition data set includes the one or more photographs; and
the imaging the initial dental configuration includes performing a three-dimensional scan of the initial dental configuration, wherein the dental condition data set includes data produced by the three-dimensional scan.

8. The method of claim 1, wherein the recording the dental condition data set includes receiving from the patient or a third party imaging data associated with the initial dental configuration.

9. The method of claim 1, wherein the producing the one or more dental aligners includes:
inputting the dental condition data set into a computer that is programmed to run a dental analysis program; and
developing a dental treatment plan;
wherein the developing the dental treatment plan is performed, at least in part, by the computer utilizing the dental analysis program and based, at least in part, on the dental condition data set.

10. The method of claim 9, wherein the developing the dental treatment plan includes:
analyzing, with the dental analysis program, the dental condition data set to produce an initial dental representation that represents the initial dental configuration of the patient; and
producing, with the dental analysis program, one or more dental treatment representations that represent a respective one or more target dental configurations.

11. The method of claim 10, wherein the producing the one or more dental treatment representations includes producing such that the initial dental representation and the one or more dental treatment representations collectively represent a sequential progression of dental configurations from the initial dental configuration of the patient to a final dental configuration of the patient.

12. The method of claim 10, wherein the producing the one or more dental aligners further includes, subsequent to the developing the dental treatment plan, creating the one or more dental aligners.

13. The method of claim 12, wherein the creating the one or more dental aligners includes, for each respective dental aligner of the one or more dental aligners:
generating a respective aligner mold that represents one of the one or more target dental configurations, and molding, with the respective aligner mold, the respective dental aligner; or
generating the respective dental aligner via at least one of an additive manufacturing process and a subtractive manufacturing process.

14. The method of claim 1, wherein the producing the one or more dental aligners includes creating at least a primary dental aligner; and wherein the providing the one or more dental aligners to the patient includes providing at least the primary dental aligner.

15. The method of claim 14, further comprising:
creating at least one secondary dental aligner that is configured to be utilized by the patient subsequent to utilizing the primary dental aligner; and
subsequent to the patient departing the service provider office, delivering the at least one secondary dental aligner to the patient.

16. The method of claim 1, further comprising, prior to the providing the one or more dental aligners to the patient; confirming that the patient will undergo treatment, wherein the confirming that the patient will undergo treatment includes examining the initial dental configuration of the patient, wherein the examining the initial dental configuration is performed by a service provider, and wherein the examining the initial dental configuration and the producing the one or more dental aligners are performed at least partially concurrently.

17. The method of claim 1, further comprising, prior to the recording the dental condition data set, acquiring the patient, wherein the acquiring the patient includes one or more of:
(i) receiving a referral from a third party;
(ii) advertising one or more aspects of the method of initiating aligner-based orthodontic treatment;
(iii) scheduling an appointment for the patient to visit the service provider office; and
(iv) receiving the patient at the service provider office without an appointment.

18. A method of initiating aligner-based orthodontic treatment of a patient, the method comprising:
recording a dental condition data set that at least partially characterizes an initial dental configuration of at least a subset of the patient's teeth, wherein the recording the dental condition data set includes imaging the initial dental configuration;
subsequent to the recording the dental condition data set, producing one or more dental aligners; and
subsequent to the producing the one or more dental aligners, providing the one or more dental aligners to the patient;
wherein the recording the dental condition data set, the producing the one or more dental aligners, and the providing the one or more dental aligners to the patient are performed at a service provider office while the patient remains at the service provider office within a total time span that is at most 3 hours.

19. The method of claim 18, wherein one or more of:
the imaging the initial dental configuration includes recording one or more X-ray images of the initial dental configuration, wherein the dental condition data set includes the one or more X-ray images;
the imaging the initial dental configuration includes recording one or more photographs of the initial dental configuration, wherein the dental condition data set includes the one or more photographs; and
the imaging the initial dental configuration includes performing a three-dimensional scan of the initial dental configuration, wherein the dental condition data set includes data produced by the three-dimensional scan.

20. A method of initiating aligner-based orthodontic treatment of a patient, the method comprising:
recording a dental condition data set that at least partially characterizes an initial dental configuration of at least a subset of the patient's teeth, wherein the recording the dental condition data set includes receiving from the patient or a third party imaging data associated with the initial dental configuration;
subsequent to the recording the dental condition data set, producing one or more dental aligners; and
subsequent to the producing the one or more dental aligners, providing the one or more dental aligners to the patient;
wherein the producing the one or more dental aligners, and the providing the one or more dental aligners to the patient are performed at a service provider office while the patient remains at the service provider office within a total time span that is at most 3 hours.
